(19) United States
(12) Patent Application Publication
(10) Pub. No.: US 2010/0158950 A1
(43) Pub. Date: Jun. 24, 2010

(54) USE OF TETANUS TOXIN TO AMPLIFY INADEQUATE VOLUNTARY MUSCLE CONTRACTION OR TO IMPROVE MUSCLE TONE IN AN ANIMAL ACTIVELY VACCINATED AGAINST THE TOXIN AND A REGIMEN FOR TREATMENT

(76) Inventor: Paul Fishman, Baltimore, MD (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/391,662

(22) Filed: Feb. 24, 2009

Related U.S. Application Data
(63) Continuation-in-part of application No. PCT/US2007/076575, filed on Aug. 22, 2007.
(60) Provisional application No. 60/823,600, filed on Aug. 25, 2006.

(51) Int. Cl. A61K 39/08; A61P 25/14; A61P 21/00

(52) U.S. Cl. 424/239.1

(57) ABSTRACT

A method of improving muscle movement, contraction and/or tone in an animal is provided. The method is carried out by administering tetanus toxin to the muscle of an animal has already been vaccinated against tetanus toxin. The toxin is administered in an amount sufficient to improve muscle movement, contraction and/or tone. The method may be used to treat patients with impaired muscle function, e.g due to compromise of the central nervous system (for example, due to stroke or spinal cord injury) or due to muscle atrophy (for example, due to immobilization after an injury). A regimen for dosage escalation of tetanus toxin is also provided.

### CROSS-REFERENCE TO RELATED APPLICATIONS

This patent application claims benefit of U.S. provisional patent application No. 60/823,600, filed on Aug. 25, 2006, and is a continuation-in-part of International patent application PCT/US07/076575, filed Aug. 22, 2007, the complete contents of both of which are incorporated herein by reference in entirety.

### STATEMENT REGARDING FEDERALLY SPONSORED RESEARCH OR DEVELOPMENT

This invention was supported, at least in part, with Veterans Affairs Merit Review Funds through the Department of Veterans Affairs. Therefore, the Government of the United States of America has certain rights in the invention.

### TECHNICAL FIELD OF THE INVENTION

The present invention relates to the use of tetanus toxin to amplify inadequate voluntary muscle contraction in an animal, which is actively vaccinated against the toxin and which has a compromised central nervous system (CNS), the use of tetanus toxin to improve muscle tone in an animal, which is actively vaccinated against the toxin, and a regimen for use in such treatments.

### BACKGROUND OF THE INVENTION

Botulinum neurotoxins have been widely used clinically as therapeutic agents. The basis of their use is the long-lasting nature of their blockade of synaptic transmission at the neuromuscular junction (NMJ) leading to reduction of muscular activity. A closely related clostridial toxin, tetanus toxin, has a somewhat different mechanism of action. Although capable of blocking neurotransmission of the NMJ, its main site of action is within the spinal cord. The toxin is bound and internalized by motor neuron terminals at the NMJ but, unlike botulinum neurotoxin, is then transported within the nerve axons to the spinal cord. The toxin is then exported out of the motor neuron cell bodies and re-internalized by surrounding pre-synaptic nerve terminals, where it inactivates local presynaptic transmission. Since synaptic transmission onto motor neurons normally is predominantly inhibitory, this inactivation of pre-synaptic transmission results in hyperactivity of affected motor neurons and unrestrained contraction (i.e., tetany) of the muscles that they innervate.

The present invention seeks to provide a method of using tetanus toxin to amplify inadequate voluntary muscle contraction in an animal, which is actively vaccinated against the toxin and which has a compromised CNS. The present invention also seeks to provide a method of using tetanus toxin to improve muscle tone in an animal, which is actively vaccinated against the toxin. In addition, the present invention seeks to provide a regimen of tetanus toxin for use in therapeutic applications. These and other objects and advantages, as well as additional inventive features, will become apparent from the detailed description provided herein.

### BRIEF SUMMARY OF THE INVENTION

The present invention provides a method of improving functional movement of a muscle in an animal in need thereof. The method comprises administering to the muscle of an animal, which is actively vaccinated against the toxin and which has a compromised CNS, an inadequate voluntary muscle contraction-amplifying amount of tetanus toxin.

The present invention also provides a method of improving the tone of a muscle in an animal in need thereof. The method comprises administering to the muscle of the animal, which is actively vaccinated against the toxin, a muscle tone-improving amount of tetanus toxin.

The present invention also provides a regimen for dosage escalation of tetanus toxin. The regimen comprises an initial dose of tetanus toxin and subsequent doses of tetanus toxin, wherein the initial dose is about 0.5 μg of tetanus toxin per muscle, each subsequent dose is at least about 2-fold higher than a preceding dose, no dose is greater than about 100 μg of tetanus toxin per muscle, and the doses are administered on a weekly basis up to about 10 weeks or until an optimal dose is determined, whichever is earlier.

### BRIEF DESCRIPTION OF THE DRAWINGS

FIG. 1. Motor score ratings for unvaccinated mice injected with tetanus toxin. Each symbol represents a single animal observation after injection. X representing animals injected with 0.2 ng; circles represent animal observations after 1.0 ng injections; triangles represented 5.0 ng injected animals.

FIG. 2. Motor score ratings for animals injected with both tetanus toxin and anti-tetanus immunoglobulin (TIG, passive immunization). Each X represents a single animal observation after injection of 1.25 μg toxin/20 IU TIG; circles represent animals injected with 2.5 μg toxin/20 IU TIG; triangles represent animals injected with 5.0 μg toxin/40 IUTIG.

FIG. 3. Motor score ratings for actively vaccinated (tetanus toxoid) mice injected with tetanus toxin. Each X represents a single animal observation after injection with 1.25 μg toxin; circles represent animal observations after 2.5 μg toxin injections; triangles represent animals injected with 5.0 μg of toxin.

FIG. 4. TA myofiber areas from normal and HI (saline- or tetanus toxin-injected) rats. Six to eight TAs per group and 100 fibers/TA were measured. *P<0.05 versus HI/Tet; **P<0.01 versus normal.

FIGS. 5A and B. Traces of A, twitch and B, tetanic tensions after nerve stimulation (in Newtons) from the TA of HI/Saline (dashed line, 3.22+/−0.24 mean and SD of three rats) and HI/Tet (solid black line, 4.77+/−0.32 mean and SD of three rats) immobilized 14 days.

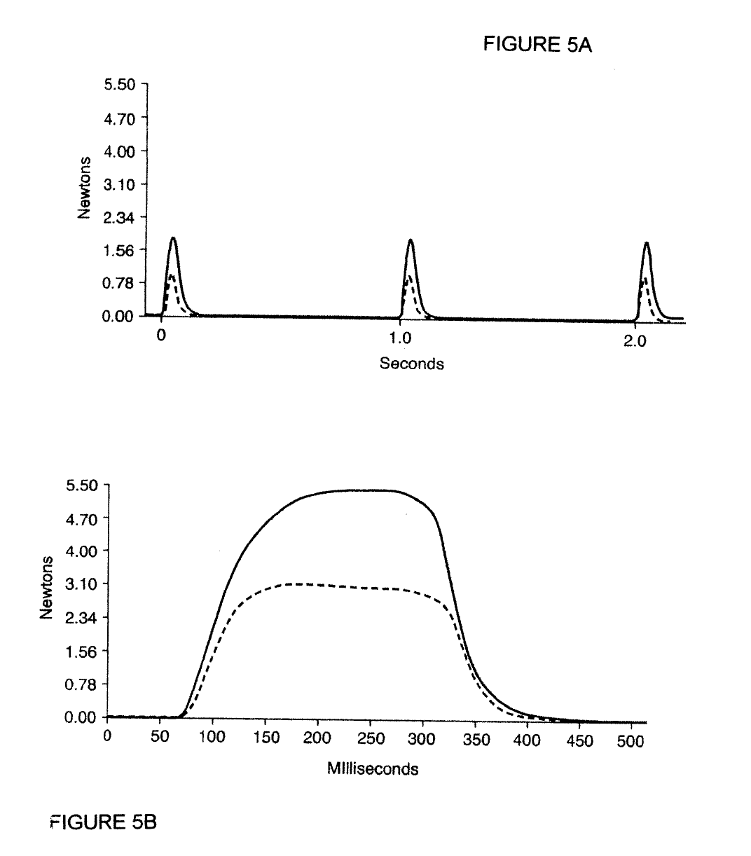

### DETAILED DESCRIPTION OF THE INVENTION

The present invention provides a method of improving functional movement of a muscle in an animal in need thereof. By “animal” is meant any animal, in particular a human, that can respond to treatment with tetanus toxin.

The method comprises administering to the muscle of an animal, which is actively vaccinated against tetanus toxin and which has a compromised CNS, an inadequate voluntary muscle contraction-amplifying amount of tetanus toxin. By “compromised” is meant that the CNS does not function normally due to, for example, disease, injury, or genetic defect. For example, the CNS can be compromised due to stroke, head injury, spinal cord injury, multiple sclerosis, and the like.

The present invention also provides a method of improving the tone of a muscle in an animal in need thereof. “Animal” is as defined above.

The method comprises administering to the muscle of the animal, which is actively vaccinated against the toxin, a muscle tone-improving amount of tetanus toxin. Improvement of muscle tone can be desired for flaccid paralysis and paresis of the face, arms, and/or legs (such as due to stroke, multiple sclerosis, Bell's palsy, brain and/or spinal cord injury), inadequate sphincter control (such as due to gastro-esophageal reflux disease (GERD), which can be treated by endoscopy using EMG guidance, or stress incontinence of the urinary sphincter), flaccid bladder, inadequate vocal cord closure and risk of aspiration, disuse atrophy (such as due to immobilization of a fractured limb), cosmetic reasons, and the like.

Tetanus toxin is commercially available from List Biological Laboratories, Inc., and Sigma-Aldrich (St. Louis, Mo.). Since the toxin provided by these suppliers is research grade, further purification or production in a GMP-certified facility is necessary for administration to humans. The tetanus toxin can be combined with a pharmaceutically acceptable carrier, such as one that is suitable for intramuscular injection. See, e.g., Remington's Pharmaceutical Sciences, Gennaro, Mack Pub. Co., 18th ed., June 1995.

The tetanus toxin is administered directly to the muscle, such as by intramuscular injection. Preferably, the tetanus toxin is injected intramuscularly under electromyographic guidance (EMG).

Dose escalation can be used to determine the optimal amount that will either amplify an inadequate voluntary muscle contraction or improve muscle tone for the longest duration of time. By “amplify” is meant to increase the size/strength of the muscle contraction during an attempt of voluntary movement. For example, an initial dose of about 0.5 μg tetanus toxin/muscle can be administered. Weekly thereafter the dose can be gradually increased, such as by about a factor of 2, up to about 100 μg tetanus toxin/muscle until an improvement in the functional movement or the tone of the treated muscle for the longest duration is observed. The dose, which provides an improvement in the functional movement or the tone of the treated muscle for the longest duration of time in a safe and tolerable manner (e.g., without significant discomfort), is the “optimal dose.” Once the optimal dose is determined by serial weekly doses (e.g., intramuscular injections), the optimal dose is administered periodically thereafter, at a time when the toxin's beneficial effect diminishes, e.g., at about 1-3 month intervals, for the duration of the condition. If, at any time, re-administration of the “optimal dose” fails to provide the expected benefit, it can be increased in the same manner as described for the initial dose escalation.

In view of the above, the present invention also provides a regimen for dosage escalation of tetanus toxin. The regimen comprises an initial dose of tetanus toxin and subsequent doses of tetanus toxin. The initial dose is about 0.5 μg tetanus toxin/muscle, and each subsequent dose is at least about 2-fold (e.g., 2-fold, 3-fold, 4-fold, 5-fold, 6-fold, 7-fold, 8-fold, 9-fold or 10-fold) higher than a preceding dose. However, no dose is greater than about 100 μg tetanus toxin/muscle. The doses are administered on a weekly basis up to about 10 weeks or until an optimal dose is determined, whichever is earlier. For example, a regimen can comprise an initial dose of about 0.5 μg tetanus toxin/muscle, and subsequent doses of about 1 μg tetanus toxin/muscle, about 2 μg tetanus toxin/muscle, and about 4 μg tetanus toxin/muscle. The dose escalation can continue up to about 10 weeks, if necessary, in order to determine the optimal dose. The optimal dose is then administered periodically thereafter, at a time when the toxin's beneficial effect diminishes, e.g., at about 1-3 month intervals, for the duration of the condition. Such regimens can be provided in the form of kits for ease of administration.

The use of the terms “a,” “an,” “the,” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate better the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

### EXAMPLES

The following example serves to illustrate the present invention. The examples are not intended to limit the scope of the invention.

### Example 1

### Local Response to Tetanus Toxin in Actively Immunized Animals

This example describes the local response to tetanus toxin in actively immunized animals.

Tetanus toxin (5 μl; 0.2 ng, 1 ng or 5 ng) was injected into the gastrocnemius muscle on one side of each of 18 unvaccinated mice. All animals in the lowest dose group developed localized tetanus of the injected limb of 1-2 weeks duration, while all animals in the higher dose groups rapidly developed generalized tetanus and were euthanized.

Another group of mice underwent active vaccination with tetanus toxin to induce protective anti-tetanus antibody titers. Vaccinated animals injected with 2-50 ng of toxin had no observable response (n=15). Vaccinated animals injected with 500 ng, 1 μg, or 5 μg of toxin (n=15) developed localized tetanus of the injected limb of variable severity and duration (up to 5 weeks), while one animal developed generalized tetanus. Response to the toxin over the first few days was highly predictive of duration and maximal severity of the motor response.

Although vaccination dramatically increases resistance to tetanus toxin, intramuscular injection of high, but easily attainable, doses of toxin can result in a prolonged, localized, spastic form of tetanus.

Therefore, it is believed that tetanus toxin can be used to enhance local motor activity in actively immunized animals with a variety of neurological conditions that result in inadequate voluntary muscle contraction.

### Example 2

### Localized Tetanus in Immunized Mice

The capacity of tetanus toxin to enhance motor neuron excitability has suggested its potential use as a therapeutic. Widespread active vaccination against tetanus in all developed countries is considered the major obstacle to clinical use of the toxin. We wished to determine the response to localized intramuscular injection of tetanus toxin in both passively and actively immunized animals as an initial exploration into the possible use of tetanus toxin as a clinical therapeutic. Unvaccinated mice underwent intramuscular injection of tetanus toxin into the gastrocnemius muscle (0.2 ng, 1 ng, or 5 ng). All animals in the lowest dose group developed only local tetanus of the injected limb. All animals in the highest dose group rapidly developed generalized tetanus. Another group of mice received anti-tetanus immunoglobulin (20-40 IU) at the time of toxin injection. These animals, although dramatically resistant to the toxin, developed predominantly local tetanus at doses of 2.5 and 5 μg. A third group of mice underwent active vaccination with tetanus toxoid to induce protective anti-tetanus immunity, then was challenged with an intramuscular injection (0.5, 1.25, 2.5, or 5 μg). All animals in this group developed local tetanus in the injected limb. The severity and duration of local tetanus was generally related to dose, but was more variable in the actively vaccinated group than in the naive or passively immunized animals. Although vaccination dramatically increases resistance to tetanus toxin, by virtue of its extremely high potency, relatively small amounts of toxin protein can produce prolonged localized tetanus even in vaccinated animals. These results suggest the possible use of tetanus toxin to enhance local motor activity in a variety of neurologic conditions.

### Introduction

Observations in humans as well as studies of experimental tetanus in animals support the concept that intramuscular injection of tetanus toxin can produce a state of hyper-excitability and over activity in a targeted population of motor neurons (Fishman, 2008). While a large number of agents including the botulinum neurotoxins are capable of reducing motor neuron or muscular activity, tetanus toxin is the only substance described that has the potential for selective enhancement of motor activity (Goonetilleke and Harris, 2004; Benecke et al, 1977). Sanders (2004) has described potential uses of tetanus toxin for a wide range of disorders of inadequate muscle tone, including sleep apnea. In the only attempt at therapy, injection with tetanus toxin into pharyngeal muscles of a single bulldog, resulted in improvement of sleep apnea without any observable adverse effects (Sasse et al, 2005).

Although one can envision the use of tetanus toxin to enhance inadequate muscle activity in a manner analogous to the widespread clinical use of the related clostridial toxin botulinum neurotoxin to suppress overactive muscles, there has been little consideration of the possible clinical utility of tetanus toxin. This disregard can be traced to the view that anti-tetanus antibodies, present in the vast majority of individuals in developed countries, would prevent the biological action of the toxin (Johnson, 1999). In contrast to the clinical use of botulinum toxin, the vast majority of individuals in developed countries have been vaccinated to prevent the occurrence of clinical tetanus. Vaccination with tetanus toxoid (formaldehyde denatured toxin) is well established to prevent clinical tetanus (Bleck, 1991). However, protection by vaccination from clinical tetanus is not absolute in humans or experimental animals. There have been several reports of clinical tetanus in vaccinated individuals even in the presence of anti-tetanus antibodies (Berger et al, 1978; Passen and Andersen, 1983; Risk et al, 1981). Local effects of tetanus toxin on the obicularis oculi muscles have also been demonstrated in rabbits who received passive immunization with anti-toxin at the time of toxin injection (Fezza, 2000). We wished to determine the extent and duration of localized clinical tetanus in animals that had undergone either passive immunization with anti-tetanus immunoglobulin or full active immunization with tetanus toxoid as a preliminary exploration toward the clinical use of the toxin in vaccinated humans.

### Experimental Procedures

Injection of tetanus toxin (List Laboratories) was performed into the gastrocnemius muscle of adult male mice (C57/BL6, male, Jackson Labs). The toxin was reconstituted in phosphate buffer and injections were made with a microliter syringe (Hamilton 30G needle) with a volume of 5 μl while under isoflurane inhalation anesthesia per an approved protocol of the IACUC of the University of Maryland. Our initial study utilized a commercially available preparation that was intended to be reconstituted at a minimum dilution of 2.5 μg/25 μl. All dilutions of tetanus toxin in our study were in excess of the manufacturer's recommendation. Our attempts to use this preparation at the highest (5 μg/5 μl) concentration resulted in visible precipitation. We also injected animals with this original preparation of toxin at a dose of 2.5 μg/5 μl. Because of concerns that the results might have been influenced by issues of toxin solubility, these results will not be reported here. The manufacturer kindly responded to our needs and provided us with a second preparation of toxin designed for reconstitution at 10 μg/25 μl. This preparation is clearly soluble at a concentration of 5 μg/5 μl, allowing us to assess this higher dose in the current study. Animals were observed daily for any signs of distress. Animals showing clear signs of generalized tetanus such as hyperextension posturing of the spine were euthanized (100 mg/kg pentobarbital). Clinical tetanus was evaluated using a motor behavior scale modified from Webster and Laurence (1963) where 5=generalized tetanus, 4=sustained spontaneous localized limb tetanus characterized by extension at the ankle and toes, 3=intermittent spontaneous limb tetanus, 2=limb tetanus consistently evoked on attempted limb movement and usually involving the entire limb, and 1=limb tetanus that involved only part of limb (usually toe extension/spreading) or that was observed inconsistently with movement. Mice were videotaped during spontaneous walking and during attempts to grasp onto a wire platform when lifted by the tail. Tapes were scored by a rater blinded to any information about the toxin injection or vaccination status of the animal.

Passively immunized mice received human hyper immune anti tetanus immunoglobulin (TIG, Talecris Biotherapeutics) by intraperitoneal (IP) injection at the time of toxin injection. Actively vaccinated mice received tetanus toxoid (Sanovi Aventis) by intraperitoneal injection with 1/50th of the recommended human dose and underwent a second (booster) vaccination 30 days later (30 days prior to tetanus toxin injection).

Sera for determination of anti-tetanus titers were obtained at the time of euthanasia. Anti-tetanus titers were performed by ELISA in a modification of a previously published protocol (Fairweather et al, 1987). Briefly, plates were coated with tetanus toxoid overnight and were then incubated with dilutions of sera from vaccinated or unvaccinated mice with a starting dilution of 1/50. Final titers were calculated by logarithmic plot of dilution versus optical density at a level of three times control (saline).

### Results

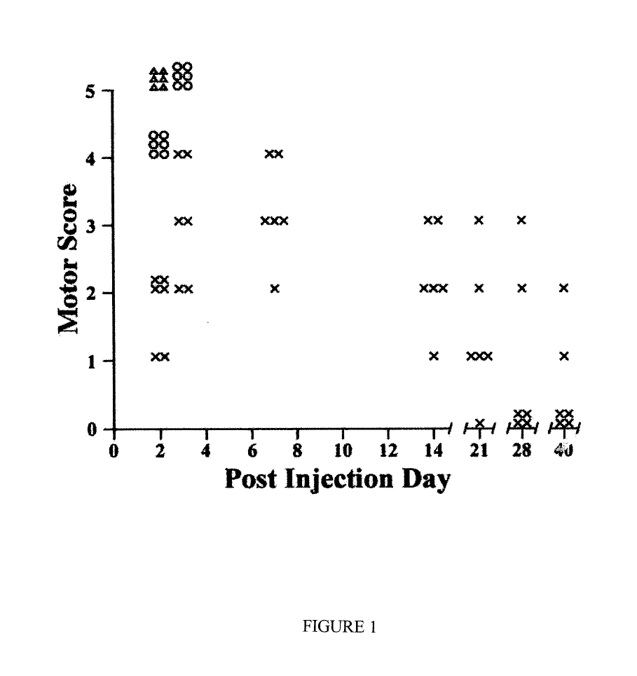

Unvaccinated (naive) mice (n=6 per dose group) were injected with one of three doses of tetanus toxin (0.2 ng, 1 ng, 5 ng). Motor responses are summarized in FIG. 1. All mice injected with the two higher doses developed generalized tetanus and died or were euthanized. Animals injected with the highest dose group developed severe localized tetanus and signs of generalized tetanus within 24 hours of toxin injection, while animals receiving the mid-dose developed signs of generalized tetanus at a slightly longer duration after injection (2-4 days). All of the animals receiving the lowest dose developed localized tetanus of at least two weeks duration, with no animal in the low dose group developing generalized tetanus.

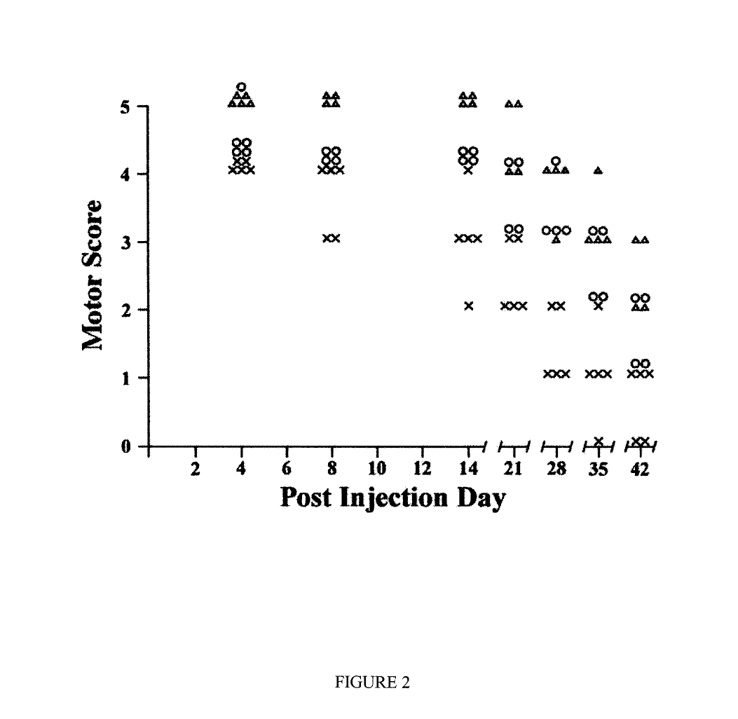

Passively Immunized Mice (n=5 per dose group) were injected with one of the following protocols: 1) 1.25 μg toxin (IM) and 20 IU TIG (IP), 2) 2.5 μg toxin with 20 IU TIG, 3) 5.0 μg toxin and 20 IU TIG and 4) 5.0 μg toxin and 40 IU TIG. All of the animals receiving the highest dose of toxin (5.0 μg) and the lower dose of TIG (20 IU) developed severe generalized tetanus within 24-48 hrs and were euthanized. The motor responses of the other three dose groups are shown in FIG. 2. All animals developed prolonged localized tetanus with some degree of generalization within the first 1-2 weeks. The dose response of animals within each group was highly consistent. Protection from the effects of toxin was substantial with animals surviving doses of toxin more than 2000 fold a uniformly lethal dose in naïve animals. Some animals in the highest dose group of both toxin and antitoxin were allowed to survive with generalized tetanus of an unusual appearance. These animals had severe but unilateral localized tetanus with curvature of the spine toward the injected side. Lack of involvement of the contra lateral hind limb was not seen in any naïve animals with comparable severity of localized motor signs.

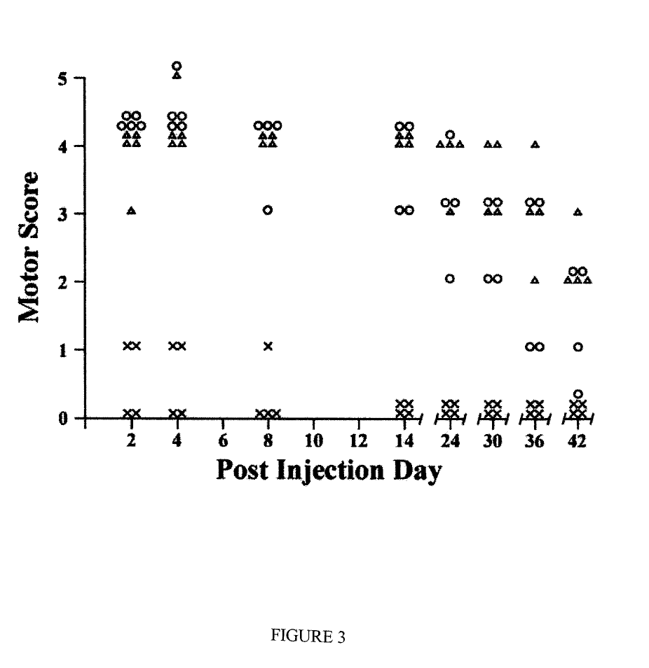

Actively Vaccinated Mice (n=5 per dose group with one anesthesia related death in the 1.25 μg group) were challenged with intramuscular injection of toxin ranging from 0.5 to 5.0 μg of toxin. Two out of five animals had brief transient localized tetanus at the lowest dose, and the motor responses of the other three groups are summarized in FIG. 3. Actively vaccinated mice were also dramatically resistant to the toxin with only one animal in each of the two highest dose groups developing generalized tetanus. Unlike the unvaccinated and passively immunized animals, actively immunized mice had a more variable response to the same dose of toxin, although this variability was less apparent at the higher dose groups, with all animals showing moderate to severe localized tetanus of a prolonged duration. All animals had protective anti tetanus antibody levels by ELISA (mean titer 1:20,000, range of titers 1:8000-1:46,000)

### Discussion

The results of this study are highly consistent with, but extend the previous literature on the action of protein neurotoxins in immune animals and humans. This is the first study evaluating the local response to tetanus toxin of animals that have been actively vaccinated against the toxin in a manner comparable to clinical practice in humans and compare them to both naive animals and animals passively immunized with TIG. In our dose ranging study, unvaccinated (naive) mice showed typical and consistent responses to the toxin ranging from local limb tetanus to generalized tetanus and death. In contrast, both passively and actively immunized animals showed dramatic protection from lethal effects of the toxin with only 2 out of 40 animals developing generalized tetanus at doses that ranged up to 5000 times a uniformly lethal dose in unvaccinated animals. Vaccinated mice were also much more resistant to the localized effects of the toxin, showing signs of local toxin action beginning at doses 500 times a uniformly lethal dose for unvaccinated mice.

These results support the concept that it is possible to produce prolonged localized tetanus with changes in motor neuron excitability and muscle tone even in a population that is either passively or actively immunized

There are potential strategies to develop a more consistent dose response to tetanus toxin injection. One strategy would be to determine the relationship between some laboratory measure of immunoresistance and the physiological effect of a specific dose of toxin. The appropriate setting to determine any correlation between anti tetanus antibody titers and the response to the toxin would be from sera obtained immediately prior to toxin injection. In addition, antibodies directed against the binding domain of the toxin are most strongly correlated with immunity to the toxin and may be used to establish such a correlation.

These observations of the time course of local and generalized tetanus provide us with very useful information. Specifically, the response to the toxin within the first few days was highly predictive of the overall response-duration profile for individual mice regardless of their vaccination status. It may be possible to consistently induce long-lasting localized tetanus without generalized tetanus using a series of repeated injections with escalating doses of toxin, followed by an observation period such as one to two weeks between injections to guide any further dose escalation.

For most protein based therapeutics the degree of immunoresistance demonstrated in this study would practically preclude any further clinical use (Chance et al, 1976; Farrell and Giovannoni, 2007). Clostridial neurotoxins are however the most potent biologic toxins known, where effective doses of tetanus toxin in vaccinated animals are increased over naive animals from the fraction of a nanogram range to only in the single microgram range, a dose that is easily attainable with currently available preparations. Although a similar situation may occur with botulinum neurotoxin, it is designed for use in immunologically naive individuals, so that development of neutralizing antibodies associated with declining efficacy make continued treatment with currently available commercial preparations impractical.

The maximal duration of the local response observed even in immune mice was over one month, and is compatible with earlier literature in animals and humans where symptoms and signs of tetanus intoxication can last from a few weeks to many months (Risk et al, 1981; Struppler et al, 1963). In agreement with earlier experimental studies and human cases, localized tetanus of at least a moderate degree is well tolerated. Mice that developed only localized tetanus showed no signs of distress, and demonstrated normal behavior except for the unusual posture of the injected limb.

These observations clearly support use of this biologic toxin with a unique mechanism of action. While a large number of agents including the botulinum neurotoxins are capable of reducing motor neuron activity, tetanus toxin is the only substance described that has the potential for selective enhancement of motor activity (Brooks et al, 1957). Studies of the clinical use of botulinum toxin in the setting of post-stroke spasticity are particularly illustrative of both the utility of a targeted anti spasticity treatment, and the need for a complimentary agent that enhances rather than reduces motor activity. Anti-spasticity treatments such as botulinum neurotoxin result in reduction of muscle activity, tone, improvement in limb posture, but infrequently result in improvement in use of the limb (Simpson et al, 2008; Sheean, 2006). Several physiologic studies support the hypothesis that inability to voluntarily activate affected muscles rather than overactivity of spastic antagonist muscles is the major source of disability in the large number of patients with so-called upper motor neuron weakness (Fellows et al, 1994; Horstman et al, 2008). Although muscles with inadequate activation can be readily identified in these patients, there is no current medical therapy to enhance their activation. Affected muscles could be specifically injected with an appropriate dose of tetanus toxin, potentially enhancing voluntary contraction of the target muscles resulting in an increase in strength. The goal of use of the toxin to enhance or amplify voluntary movement has implications for the large number of patients with motor disability due to conditions such as brain or spinal cord injury, stroke or multiple sclerosis.

This study supports the conclusion that immunity against tetanus toxin can be overcome with a sufficiently high dose of toxin. This conclusion places the previous assumption, that widespread human vaccination against the toxin would make clinical use of tetanus toxin unfeasible, under serious challenge, and suggests the clinical utility of tetanus toxin for a wide range of motor disorders.

### Example 3

### Disuse Muscle Atrophy: An Initial Target for Tetanus Toxin Therapy

Although tetanus toxin has clear therapeutic potential for a wide range of indications, most are persistent conditions that would likely require repeated series of toxin treatments in a manner similar to current botulinum neurotoxin treatment. A condition of limited duration, in which a single course of treatment could have a significant impact is disuse muscle atrophy.

Muscle atrophy after limb immobilization is a major obstacle in the rehabilitation of patients after limb trauma or surgery. In spite of current therapy, a large number of patients have prolonged and significant loss of muscle mass and strength. This is particularly true for fragile and cognitively impaired patients who are unable to comply with early and intensive exercise rehabilitation protocols. A new form of therapy that could prevent or reverse muscle atrophy associated with immobility would significantly compliment current rehabilitation regimens for patients with limb trauma.

We have performed a pilot experiment in unvaccinated rats to assess the capacity of tetanus toxin to prevent disuse atrophy. To induce muscle atrophy, one hindlimb was surgically immobilized (HI) at the knee. At the time of immobilization, the tibialis anterior (TA) muscle was injected with saline (HI/Saline) or tetanus toxin (HI/Tet; 2.5 ng/5 ml). TAs from normal rats were injected with saline (N/Saline) as a control. After 2 weeks, the muscles were removed, weighed, and processed for histology.

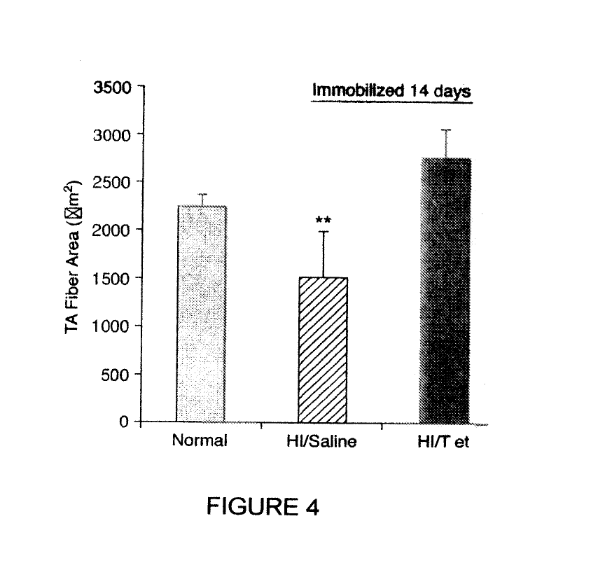

As expected, hindlimb immobilization produced substantial atrophy in the saline injected TAs. HI/Saline TA muscles had a wet weight normalized to body weight of 1.71±0.16 (mean±SD; N=6), which was significantly different from wet weight of TAs from normal rats that were injected with saline but not immobilized (2.42±0.30; P<0.001; N=6). Most promising, TAs that were immobilized and injected with tetanus toxin (2.20±0.24; P<0.01; N=7) were significantly larger than the immobilized, saline injected TAs, the same muscles used to determine fiber cross-sectional areas. FIG. 34-4A shows that the fiber cross-sectional areas of the HI/Saline TAs (1522±462 mm2) were smaller than those from the HI/Tet (2759±306 mm2; P<0.05) and N/Saline (2249±125 mm2; P<0.01) groups.

With the goal of muscle rehabilitation being the return of function, we were interested in determining whether the HI/Tet rat TAs could produce more force than the HI/Saline TAs. Our preliminary results suggest that they do. FIG. 34-4b shows representative tracings of twitch and tetanic (Po) tensions obtained from HI/Saline (N=3) and HI/Tet (N=3) rats. The TAs from HI/Tet rats produced 63% greater twitch and 48% greater tetanic tension than those from the HI/Saline rats.

All of the tetanus toxin-injected animals showed clinical signs of localized tetanus of the injected muscle. All of the injected animals otherwise showed normal behavior, with no signs of distress or of generalized tetanus. These results are encouraging for the further development of tetanus toxin as a treatment to prevent disuse muscle atrophy. This condition with its well-validated animal model, expected effects from only a single course of treatment, and its high clinical importance makes it a logical target for tetanus toxin therapy

### Example 4

### Uses of Tetanus Toxin to Enhance Inadequate Muscle Activity

Tetanus toxin is used to enhance inadequate muscle activity in a manner analogous to the clinical use of botulinum neurotoxin to suppress excessive activity. Current state-of-the-art evaluation of patients with motor deficits from conditions such as stroke or cerebral palsy involves both clinical and electrophysiologic analysis of attempts at functionally important movements such as grasping and walking. In this manner, one can discover the pattern of muscle activation during these acts and determine the degree of inappropriate overactivation, as well as inadequate activation of specific muscles. For muscles in which this analysis determines that overactivity of particular muscles as occurs in spasticity interferes with normal posture and movement, those muscles are typically injected with botulinum neurotoxin (Esquenazi and Mayer, 2004). Injection of botulinum neurotoxin can result in reduction of muscle activity, tone, improvement in limb posture, and to some extent, improvement in use of the limb. Although several strategies, including the use of botulinum neurotoxin, are available to treat spasticity, their capacity to improve motor function and voluntary movement is limited (Sheean 2006).

This limitation is explained by studies in patients after stroke demonstrating that rather than spasticity, inadequate voluntary muscle activation is the major source of weakness and motor disability (Fellows et al. 1994). The reduced population of surviving cortical motor neurons after stroke and other forms of brain injury generates a reduced voluntary motor signal, resulting in minimal activation of the target spinal motor neurons and inadequate muscle contraction. Although the many muscles with inadequate activation can be identified in patients affected by conditions such as stroke, MS, brain or spinal cord injury, there is no current medical therapy to enhance their activation. These muscles are injected with an appropriate dose of tetanus toxin which causes enhancement of voluntary contraction of the target muscles, resulting in an increase in strength. Like botulinum neurotoxin, tetanus toxin has a long duration of action, with the opportunity for repeated treatments and long-term benefit. Tetanus toxin may be the only substance with the potential for selective enhancement of muscle activation in brain- or spinal cord-injured patients, suggesting its use in chronic conditions such as weakness or hypotonia after stroke.

### REFERENCES

Benecke R, Takano K, Schmidt J, Henatsch H (1977) Tetanus toxin induced actions on spinal Renshaw cells and Ia-inhibitory interneurons during development of local tetanus in the cat. Exp. Brain Res 27:271-286
Berger S A Cherubin, C. E., Nelson, S., Levine, L (1978) Tetanus despite pre-existing anti-tetanus antibody. JAMA 240:769-770
Bleck T P (1991) Tetanus: pathophysiology, management, and prophylaxis. Dis Mon 37:545-603
Brooks V B, Curtis D R, Eccles J C (1957) The action of tetanus toxin on the inhibition of motoneurons. J Physiol 135:655-672
Chance R E, Root M A, Galloway J A (1976) The immunogenicity of insulin preparations. Acta Endocrinol. Suppl (Copenh) 205:185-198
Esquenazi A, Mayer N H. Instrumented assessment of muscle overactivity and spasticity with dynamic polyelectromyographic and motion analysis for treatment planning. Am J Phys Med Rehabil. 2004; 83(suppl):S19-S29.
Fairweather N F, Lyness V A, Maskell, D J (1987) Immunization of mice against tetanus with fragments of tetanus toxin synthesized in Escherichia coli. Infect Immun 55:2541 [symbol] 2545
Farrell R A, Giovannoni G (2007) Measuring and management of anti-interferon beta antibodies in subjects with multiple sclerosis. Mult Scler 13:567-577
Fellows S J, Kaus C, Thilmann, A F (1994) Voluntary movement at the elbow in spastic hemiparesis. Ann Neurol 36:397-407
Fezza J P, Howard J, Wiley R, Wesley R E, Klippenstein K, Dettbarn W (2000) The effects of tetanus toxin on the orbicularis oculi muscle. Ophthal Plast Reconstr Surg 16:101-113
Fishman P S (2008) Tetanus toxin. In: Botulinum Toxin: Therapeutic Clinical Practice and Science, Jankovik J, Albanese A, Atassi M Z, Dolly J O, Hallett M, Eds. Elsevier (In press). Goonetilleke A, Harris J B (2004) Clostridial neurotoxins. J Neurol Neurosurg Psychiatry 75 Suppl 3:iii 35-39
Horstman A M, Beltman M J, Gerrits K H, Koppe P, Janssen T W, Elich P, de Haan A (2008) Intrinsic muscle strength and voluntary activation of both lower limbs and functional performance after stroke. Clin Physiol Funct Imaging 28:251-61
Johnson E A (1999) Clostridial toxins as therapeutic agents: benefits of nature's most toxic proteins. Ann Rev Microbiol 53:551-575
Passen E L, Andersen B R (1986) Clinical tetanus despite a protective level of toxin-neutralizing antibody. JAMA 255:1171-1173
Risk W S, Bosch E P, Kimura J, Cancilla P A, Fischbeck K H, Layzer R B (1981) Chronic tetanus: clinical report and histochemistry of muscle. Muscle Nerve 5:363-366
Sanders I (2004) Method for using tetanus toxin for beneficial purposes in animals (mammals). US Patent Application Publication. Pub No. US2004/248188A1 December 9
Sasse A, Conduit R, Ryan D, Woods W, Tucker A (2005) A pharmacotherapy for obstructive sleep apnea. Sleep 28:1015-1016
Sheean G (2006) Botulinum toxin treatment of adult spasticity: a benefit-risk assessment. Drug Saf 29:31-48
Simpson D M, Gracies J M, Graham H K, Miyasaki J M, Naumann M, Russman B, Simpson L L, So Y (2008) Therapeutics and Technology Assessment Subcommittee of the American Academy of Neurology. Assessment: Botulinum neurotoxin for the treatment of spasticity (an evidence-based review): report of the Therapeutics and Technology Assessment Subcommittee of the American Academy of Neurology. Neurology 70:1691-8
Struppler A, Strupler E, Adams R D (1963) Local tetanus in man. Its clinical and neurophysiological characteristics. Arch Neurol 8:162-78
Webster R A, Laurence D R (1963) The effect of antitoxin on fixed and free toxin in experimental tetanus. J Pathol Bacteriol 86:413-420

Preferred embodiments of this invention are described herein, including the best mode known to the inventors for carrying out the invention. It should be understood that the illustrated embodiments are exemplary only, and should not be taken as limiting the scope of the invention.

What is claimed is:

1. A method of improving functional movement of a muscle in an animal in need thereof, wherein the animal is vaccinated against tetanus toxin, and wherein the muscle otherwise displays inadequate voluntary muscle contraction, comprising the step of
administering to the muscle of the animal an amount of tetanus toxin sufficient to cause muscle contraction, whereupon functional movement of the muscle in the animal is improved.

2. The method of claim 1, wherein the central nervous system (CNS) of the animal is compromised.

3. The method of claim 2, wherein the animal's CNS is compromised due to stroke, head injury, spinal cord injury, or multiple sclerosis.

4. The method of claim 1, wherein voluntary contraction of the muscle is impaired due to atrophy.

5. The method of claim 1, wherein the tetanus toxin is administered intramuscularly.

6. The method of claim 5, wherein the tetanus toxin is administered intramuscularly with electromyographic guidance (EMG).

7. The method of claim 1, wherein increasing amounts from 0.5 μg tetanus toxin/muscle to 100 μg tetanus toxin/muscle are administered weekly for up to 10 weeks to determine an optimal dose, after which the optimal dose is administered every 1-3 months.

8. The method of claim 7, wherein an initial dose of tetanus toxin is 0.5 μg tetanus toxin/muscle.

9. The method of claim 8, wherein each subsequent dose which is administered is 2-fold higher than a preceding dose.

10. The method of claim 7 wherein the optimal dose is administered monthly.

11. A method of improving tone of a muscle in an animal in need thereof, wherein the animal is vaccinated against tetanus toxin, comprising,
administering to the muscle of the animal a muscle tone-improving amount of tetanus toxin.

12. The method of claim 11, wherein the tetanus toxin is administered intramuscularly.

13. The method of claim 12, wherein the tetanus toxin is administered intramuscularly with EMG.

14. The method of claim 11, wherein increasing amounts from 0.5 μg tetanus toxin per muscle to about 100 μg tetanus toxin per muscle are administered weekly for up to 10 weeks to determine an optimal dose, after which the optimal dose is administered every 1-3 months.

15. The method of claim 12, wherein an initial dose of tetanus toxin is 0.5 μg tetanus toxin/muscle.

16. The method of claim 15, wherein subsequent doses administered after the initial dose are 2-fold higher than each preceding dose.

17. The method of claim 14 wherein the optimal dose is administered monthly.

18. A regimen for dosage escalation of tetanus toxin comprising an initial dose of tetanus toxin and subsequent doses of tetanus toxin, wherein the initial dose is 0.5 μg tetanus toxin per muscle, each subsequent dose is at least 2-fold higher than a preceding dose, no dose is greater than 100 μg tetanus toxin per muscle, and the doses are administered on a weekly basis up to 10 weeks or until an optimal dose for improving muscle movement or tone is determined, whichever is earlier.

19. The method of claim 18, wherein an initial dose of tetanus toxin is 0.5 μg tetanus toxin/muscle.

20. The method of claim 19, wherein the subsequent dose is 2-fold higher than the preceding dose.
